(19) United States
(12) Patent Application Publication
(10) Pub. No.: US 2016/0367505 A1
(43) Pub. Date: Dec. 22, 2016

(54) PROTOTYPICAL ANALGESIC PROFILE OF N-STEARYL DOPAMINE (NS-DA)

(71) Applicant: Alexandre Vamvakides, Ymittos Attikis (GR)

(72) Inventor: Alexandre Vamvakides, Ymittos Attikis (GR)

(21) Appl. No.: 14/744,562

(22) Filed: Jun. 19, 2015

(51) Int. Cl. A61K 31/16

(52) U.S. Cl. CPC A61K 31/16

(57) ABSTRACT

The present invention discloses the prototypical profile of N-Stearyl dopamine (NS-DA) against the anticancer drugs induced neuropathic pain (ADINP). Indeed, NS-DA exhibited high analgesic activity against the Paclitaxel induced neuropathic sensitization (PINS), in mice, and also decrease of brain dopamine (DA) turnover and of motility, in rats and mice, i.e., the exactly opposite effects of those induced by the μ-opioid receptor agonists (μ-ORAs). Thus, the above properties of NS-DA allow the beneficial synergy of NS-DA and μ-ORAS, with NS-DA reinforcing the analgesic properties of μ-ORAs and protecting against the opioid dependence and the neurotoxicity of μ-ORAs, coming from the increase of the brain DA turnover induced by the μ-ORAs.

### BACKGROUND OF THE INVENTION

### Field of the Invention

The present invention relates to a prototypical analgesic profile of N-stearyl dopamine (NS-DA) for use in connection against the neuropathic pain induced by the anticancer drugs.

### Description of the Prior Art

Prototypical analgesic profile of N-stearyl dopamine (NS-DA) is desirable for treating against anticancer drugs induced neuropathic pain (ADINP). Pain can be generally categorized in terms of the origin to three types: nociceptive pain, neuropathic pain, and psychogenic pain. Nociceptive pain occurs by the mediation of nociceptors when a tissue is damaged or noxious stimulation that may damage a tissue is applied to the living body. Neuropathic pain is caused by primary damage of the nervous system or a function disorder of the nervous system or induced by the damage or disorder. Neuropathic pain is caused by damage of the peripheral nervous system or the central nervous system. Psychogenic pain is a type of pain which exhibits no lesion sufficient to cause pain and which cannot be elucidated anatomically.

Typical examples of neuropathic pain include pain in diabetes patients and in alcoholic patients, adverse side effects of anticancer agents (e.g., cisplatin, paclitaxel, vincristine), postoperative pain, phantom pain, postherpetic pain, trigeminal neuralgia, and central neuralgia.

ADINP is the most common treatment limiting problem encountered by patients receiving taxane (paclitaxel), vinca alcaloids (vincristine) or platin based (cisplatin, oxaliplatine) chemotherapy. It improves or completely resolves in 51% of patients, but becomes chronic in the remainder, affecting the quality of life and return to productivity in cancer survivors. Therefore, peripheral neuropathy is the chief dose-limiting side effect associated with all three major classes of frontline drugs that are used against all of the most common types of cancer. Patients experience numbness, tingling, burning pain and sensory-motor impairments. The cause of this neuropathic pain syndrome is unknown and unfortunately no really effective pharmacological options to its treatment are available.

Clinically, the analgesic treatments administered against ADINP are Gabapentin, Pregabalin and mu-opioid receptor agonists (μ-ORAs) such as morphine, oxycodone or fantanyl. Usually, Gabapentin or Pregabalin have modest effects against ADINP and they are replaced by μ-ORAs or, more frequently, associated with μ-ORAs. Although the μ-ORAs, alone or in association with Gabapentin or Pregabalin, have significant (but not satisfying) antinociceptive effects against ADINP, they exhibited several adverse effects: nausea, vomiting, constipation, respiratory depression and also the most serious problem of opioid dependence, the latter being a limiting factor in the amelioration of the analgesia by augmenting the doses of μ-ORAs. It is widely admitted that the activation of the brain nigro-striatal and meso-limbic dopaminergic systems have a prominent role in the opioid dependence, with the first system being probably more important [1-4].

Therefore, a need exists for a new and improved prototypical analgesic profile of N-stearyl dopamine (NS-DA) that can be used against the neuropathic pain induced by the anticancer drugs. In this regard, the present invention substantially fulfills this need. In this respect, the prototypical analgesic profile of N-stearyl dopamine (NS-DA) according to the present invention substantially departs from the conventional concepts and designs of the prior art, and in doing so provide an apparatus primarily developed for the purpose against the neuropathic pain induced by the anticancer drugs.

### BRIEF SUMMARY OF THE INVENTION

In view of the foregoing disadvantages inherent in the known types of analgesic treatments against ADINP now present in the prior art, the present invention provides an improved prototypical analgesic profile of N-stearyl dopamine (NS-DA), and overcomes the above-mentioned disadvantages and drawbacks of the prior art. As such, the general purpose of the present invention, which will be described subsequently in greater detail, is to provide a new and improved prototypical analgesic profile of N-stearyl dopamine (NS-DA) and method which has all the advantages of the prior art mentioned heretofore and many novel features that result in a prototypical analgesic profile of N-stearyl dopamine (NS-DA) which is not anticipated, rendered obvious, suggested, or even implied by the prior art, either alone or in any combination thereof.

To attain this, the present invention essentially comprises a method of using a compound N-Stearyl dopamine (NS-DA) for the preparation of pharmaceuticals with analgesic properties against anticancer drugs induced neuropathic pain (ADINP).

Principally, the compound can be used in association with μ-opioid receptor agonists (μ-ORAs) for protective activity against psychological dependence induced by the μ-ORAs.

The compound can further include at least one pharmaceutically acceptable excipient.

There has thus been outlined, rather broadly, the more important features of the invention in order that the detailed description thereof that follows may be better understood and in order that the present contribution to the art may be better appreciated.

The invention may also include a daily administration dosage of the compound to a patient at 10 to 100 mgs. There are, of course, additional features of the invention that will be described hereinafter and which will form the subject matter of the claims attached.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. In this respect, before explaining the current embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

It is therefore an object of the present invention to provide a new and improved prototypical analgesic profile of N-stearyl dopamine (NS-DA) that has all of the advantages of the prior art analgesic treatments against ADINP and none of the disadvantages.

It is another object of the present invention to provide a new and improved prototypical analgesic profile of N-stearyl dopamine (NS-DA) that may be easily and efficiently manufactured and marketed.

These together with other objects of the invention, along with the various features of novelty that characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a graph showing the anticancer drugs induced neuropathic pain (ADINP) therapeutic effect of single administration of compounds (A1, A2 and A3) associated with morphine.

### DETAILED DESCRIPTION OF THE INVENTION

An embodiment of the present invention discloses the prototypical profile of N-stearyl dopamine (NS-DA) against the anticancer drugs induced neuropathic pain (ADINP). NS=DA, as referred to in the present invention, may include compounds having the general formula A

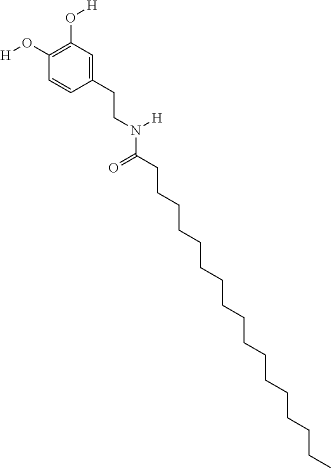

NS-DA was synthesized and studied pharmacologically in the early ninety's. The NS-DA neuro-pharmacological profile was characterized by hypomotility and decrease of the brain dopamine turnover, in mice and rats. As the above neuro-pharmacological properties of NS-DA were exactly the opposite of those induced by the μ-ORAs (the latter exhibiting hyperlocomotion and increase of the brain DA turnover), testing of NS-DA in the ADINP model of Paclitaxel induced pain sensitization in the formalin test of pain, on mice and it obtained a high analgesic activity of NS-DA against the Paclitaxel induced neuropathic sensitization (PINS).

### EXAMPLE 1

NS-DA (40 mg/kg, intraperitoneal: ip), dissolved and administrated in Cremophor.EL (compound A1) suppressed the PINS by 94.8%, in the 35-40 mn central sensitization phase, in the formalin induced pain, when the NS-DA (40 mg/kg, ip) group was compared to the Cremophor.EL group (control), 1 hour after administrations.

### EXAMPLE 2

When NS-DA was administered per os (po), in Tween-80 (5%) suspension (compound A2), PINS diminished by 39%, 62% and 76% at, respectively 1, 2 and 4 hours after administrations, when compared to the Tween-80 (5%) controls.

### EXAMPLE 3

Finally, co-administration of low doses of morphine (3 mg/kg, sc) and NS-DA (10 mg/kg, ip, in Cremophor) (compound A3) suppressed the PINS in the 35-40 mn central sensitization phase (decrease by 83.6%), where NS-DA (10 mg/kg, ip) was inactive and morphine (3 mg/kg, sc) induced only 37% decrease of PINS.

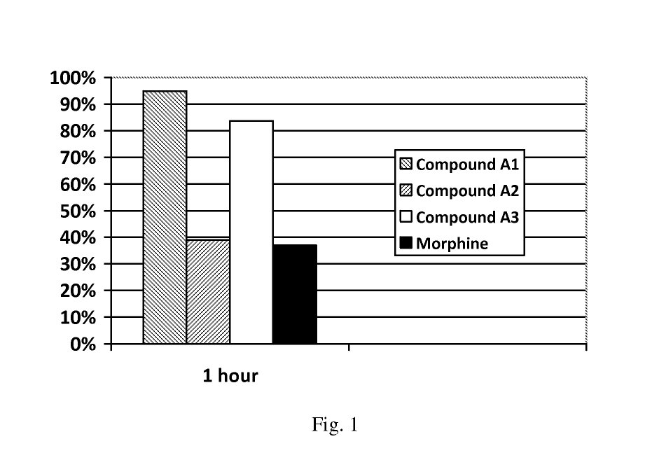

The therapeutic effects of compounds (A1, A2 and A3) can be seen in FIG. 1, illustrated the results of administering the compounds to treat against ADINP.

It is noteworthy that NS-DA at 10-100 mg/kg (ip) exhibited the opposite effects, on locomotion and brain DA turnover to those induced by μ-ORAs, effects which were characterized as originating from the unique and selective agonistic activity of NS-DA on the extrasynaptic DA receptors in the substantia nigra (SN) and ventral tegmental area (VTA), for the decrease of the DA turnover, and in the dorsal root ganglia (DRG), the latter conferring to NS-DA a prototypical profile of analgesic drug against ADINP. It could be co-administered with the μ-ORAs amplifying their analgesic activity and, more importantly, protecting against the opioid dependence of the μ-ORAs.

Concerning the gender differences in the analgesic effect of NS-DA on the PINS of 35-40 mn and 55-60 mn central phases of the formalin induced pain, in comparison to the phase of the 0-5 mn acute pain induced by formalin, the NS-DA analgesic effect was more prominent in the 35-40 mn sensitization phase and was similar for males and females (about 95% decrease) while, in the 0-5 mn acute pain phase, the NS-DA analgesic effect was not significant in male mice and high (65% decrease) in females. Similarly in the 55-60 mn sensitization phase the effect of NS-DA was higher in female mice (95% decrease) compared to its effect on males (58% decrease). These differences originated from the more important pain sensitivities of control female mice concerning the first, acute pain, phase (0-5 mn) and also the sensitization phases 35-40 mn and 55-60 mn, in agreement with the experimentally (mice, rats) or clinically (human) observed gender differences in pain (acute or neuropathic) sensitivity, with females being more sensitive [9-10].

Finally, concerning the putative role of excessive release or accumulation of DA in the CNS neurotoxicity, in the early ninety's the author signaled the rational of glutamatergic potentiation by the redox perturbation originating by the above excessive DA release (as observed with the μ-ORAs) or DA accumulation (as observed in brain ischemias). Recently, this problem was confirmed and also envisaged in relation to morphine. NS-DA, by its robust inhibition of the CNS DA turnover is, therefore, indicated also against the μ-ORAs excessive DA turnover, their oxidative stress triggered in the CNS and the resulting neurotoxicity, which constitute the final stage of the opioid dependence [11-15].

While embodiments of the prototypical analgesic profile of N-stearyl dopamine (NS-DA) have been described in detail, it should be apparent that modifications and variations thereto are possible, all of which fall within the true spirit and scope of the invention. And although against the neuropathic pain induced by the anticancer drugs have been described, it should be appreciated that the prototypical analgesic profile of N-stearyl dopamine (NS-DA) herein described is also suitable for possible other treatments.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### REFERENCES

1) Kuschinsky K., Arzneimittelforschung, 1976, 26, (4): 563-7.
2) Oka T., Hosoya E., Jpn. J. Pharmacol., 1976, 26 (5): 615-9.
3) Mikkola J. A., Honkanen A., et al., Pharmacol. Biochem. Behav., 2000, 67 (4): 783-91.
4) Mori T., Kanbara T., et al., J. Pharmacol. Sci., 2014, 126 (1): 47-55.
5) Kolocouris N., Foscolos G. B., et al., Ann. Pharm. Fr., 1991, 49 (2): 99-110.
6) Antoniou K., Vamvakides A., et al., Prog. Neuropsychopharmacol. Biol. Psychiatry, 1996, 20 (1): 111-9.
7) Matsumoto M., Inoue M., et al., J. Pharmacol. Exp. Ther., 2006, 318 (2): 735-40.
8) Riganas S., Papanastasiou I., et al., Bioorg. Med. Chem., 2012, 20 (10): 3323-31., Med. Chem., 2012, 8 (4): 569-86, J. Med. Chem., 2012, 55 (22): 10241-61.
9) Bartley E. J., Fillingim R. B., Br. J. Anaesth., 2013, 111 (1): 52-8.
10) Naji-Esfahani H., Vaseghi G., et al., Lab. Anim. 2015,
11) Vamvakides A., Ann. Pharm. Fr., 1992, 50 (5-6): 277-89.
12) Vamvakides A. Ann. Pharm. Fr., 1993, 51 (1): 8-15.
13) Nakagawa T., Kaneko S., J. Pharmacol. Sci., 2008, 106 (1): 2-8.
14) Aluf Y., Vaya J., et al., Neuropharmacology, 2013, 65: 48-57.
15) Skrabalova J., et al., Mini. Rev. Org. Chem. 2013, 10 (4): 367-72.

What is claimed as being new and desired to be protected by Letters Patent of the United States is as follows:

1. A method of using N-Stearyl dopamine (NS-DA) to treat against neuropathic pain, said method comprises administering to an individual suffering from neuropathic pain an effective amount of NS-DA.

2. The method according to claim 1, wherein said neuropathic pain is anticancer drugs induced neuropathic pain (ADINP).

3. The method according to claim 2, wherein said NS-DA is prepared as a pharmaceutical composition having analgesic properties against ADINP.

4. The method according to claim 3, wherein said pharmaceutical composition is administered to the individual in an amount between 10 to 100 mgs, daily.

5. The method according to claim 4, wherein said pharmaceutical composition is in association with μ-opioid receptor agonists (μ-ORAs) for protective activity against psychological dependence induced by said μ-ORAs when used against ADINP.

6. A method of using N-Stearyl dopamine (NS-DA) to treat against anticancer drugs induced neuropathic pain (ADINP), said method comprising the steps of:
preparing a pharmaceutical composition comprising N-Stearyl dopamine (NS-DA), said pharmaceutical composition having analgesic properties against ADINP; and
administering to an individual suffering from neuropathic pain an effective amount of said pharmaceutical composition.

7. The method according to claim 6, wherein said pharmaceutical composition further comprising at least one pharmaceutically acceptable excipient.

8. A method of dosing for protective activity against anticancer drugs induced neuropathic pain (ADINP) with N-Stearyl dopamine (NS-DA), said method comprising the steps of:
preparing a compound of N-Stearyl dopamine (NS-DA); and
administering said compound to an individual in an amount between 10 to 100 mgs, daily.

9. The method according to claim 8, wherein said compound further comprising at least one pharmaceutically acceptable excipient.

10. The method according to claim 8 further comprising the step of using said compound in association with μ-opioid receptor agonists (μ-ORAs) for protective activity against psychological dependence induced by said μ-ORAs.

11. The method according to claim 10 further comprising the step of interacting said compound with said μ-ORAs so that said compound reinforces analgesic properties of said μ-ORAs and protects the individual against the ADINP of said μ-ORAs.

12. The method according to claim 11, wherein said compound is configured to protect the individual against an opioid dependence of said μ-ORAs.

13. The method according to claim 11, wherein said compound is configured to protect the individual against neurotoxicity of said μ-ORAs.
